(19) United States
(12) Patent Application Publication
(10) Pub. No.: US 2017/0088822 A1
(43) Pub. Date: Mar. 30, 2017

(54) Crystalline Structure of FABI from Burkholderia Pseudomallei

(71) Applicants: Narasimha Rao Krishnamurthy, Bangalore (IN); Anirudha Lakshminarasimhan, Bangalore (IN); Thomas Antony, Bangalore (IN); Hosahalli S. Subramanya, Bangalore (IN)

(72) Inventors: Narasimha Rao Krishnamurthy, Bangalore (IN); Anirudha Lakshminarasimhan, Bangalore (IN); Thomas Antony, Bangalore (IN); Hosahalli S. Subramanya, Bangalore (IN)

(73) Assignee: AURIGENE DISCOVERY TECHNOLOGIES LIMITED, Bangalore (IN)

(21) Appl. No.: 14/871,383

(22) Filed: Sep. 30, 2015

(51) Int. Cl. C12N 9/02; C12Q 1/26; C40B 30/02; C07D 471/04; G06F 19/12

(52) U.S. Cl. CPC C12N 9/001; C07D 471/04; G06F 19/12; C40B 30/02; C12Q 1/26; C12Y 103/01009; G01N 2333/90206

(57) ABSTRACT

The present invention relates to drug targets for Burkholderia pseudomallei. The invention provides a crystalline polypeptide derived from Burkholderia pseudomallei comprising the amino acid sequence set forth in SEQ ID NO: 1. Also provided are methods for co-crystallizing a binary enoyl-acyl carrier protein reductase (FabI) with a potential inhibitor of an FabI activity and for identifying an inhibitor of an activity of enoyl-acyl carrier protein reductase (FabI). A representative example of such a crystalline structure is a BpmFabI:AFN-1252 complex.

### BACKGROUND OF THE INVENTION

Field of the Invention

The present invention relates to the crystalline structure of FabI from organism Burkholderia pseudomallei (BpmFabI). Specifically, the present invention relates to the crystalline structure of FabI from organism Burkholderia pseudomallei in binary complex with AFN-1252 in the absence of cofactors. This invention also relates to use of crystalline FabI in the rational design and development of new modulators for BpmFabI.

Description of the Related Art

Fatty acid biosynthesis (or Fab) is an essential metabolic process for all living organisms. It is used to synthesize the metabolic precursors for membrane phospholipids in the cell wall. Fatty acids are synthesized by mammals (using enzyme FAS I) and bacteria (using enzyme FAS II) via substantially different biosynthetic mechanisms, thus providing the possibility of bacteria-specific drug targeting. Indeed, inhibitors targeting the various stages of the fatty acid biosynthetic pathway have been investigated as novel anti-bacterial agents. Broadly, the pathway of saturated fatty acid biosynthesis (FAB) is more or less similar in all organisms, however, the fatty acid synthase (FAS) enzymatic biosynthesis systems vary considerably with respect to their structural organization. Mammalian fatty acid synthesis (FAS-I) employs a multifunctional enzyme complex in which all enzymatic activities reside on a single polypeptide. In contrast, bacterial fatty acid synthesis (FAS-II) elongation cycle utilizes several distinct monofunctional enzymes with activity pertaining to respective eenzyme peptides effecting fatty acid chain elongation and ultimately cell membrane production. Enoyl acyl carrier protein reductase (FabI) is the component of FAS-II that catalyzes the final reaction in the enzymatic sequence. Hence, there appears to be considerable scope for the selective inhibition of the bacterial FAS system enzymes by exploring newer anti-bacterial agents.

FabI acts as an enoyl-ACP reductase in the final step of the reactions involved in each cycle of bacterial fatty acid biosynthesis. Further rounds of this cycle, adding two carbon atoms per cycle, eventually lead to palmitoyl-ACP (16-Carbon), and subsequently the cycle is blocked largely due to feedback inhibition of FabI by palmitoyl-ACP.

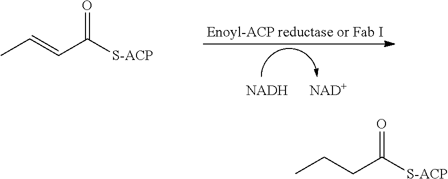

Thus, FabI is among one of the major biosynthetic enzyme and appears to be a key moderator in the overall bacterial fatty acid biosynthetic pathway. Therefore, FabI may be one of the meaningful targets for acquiring anti-bacterial role.

Fatty acid biosynthetic pathway is being actively pursued for the development of antibacterial agents. Since FabI catalyzes the last step in fatty acid chain elongation and play a determinant role, it was identified as a specific cellular target for therapeutic intervention. FabI enzymes from Gram-positive and Gram-negative bacteria have significant sequence and structural similarity. Hence, an understanding of the mechanism of binding of the inhibitors to FabI and structural insights from FabI-inhibitor complexes from Gram-negative bacteria will be useful in designing new inhibitors.

Melioidosis is an infectious disease caused by the Gram-negative organism Burkholderia pseudomallei (B. pseudomallei) that affects both humans and animals. This infection is recognized as an important health problem in southeast Asia and tropical northern Australia. Clinical manifestations of naturally occurring melioidosis include pneumonia with or without septicemia or a localized infection involving the skin and soft tissue organs. Chronic disease might also occur; symptoms of which mimic those of tuberculosis, and it is clinically challenging to distinguish these two diseases. The treatment options include ceftazidime or carbapenem as IV dosing for two weeks during the initial intensive phase of therapy followed by twelve weeks of oral therapy. Drug resistance has already been reported with current treatment, suggesting that an effective treatment of B. pseudomallei infection will be a challenging task. Moreover, B. pseudomallei are intrinsically resistant to several classes of antibiotics due to expression of resistance determinants such as beta-lactamase and multidrug efflux pumps.

Type II bacterial fatty acid synthesis (FASII) is an essential pathway for both Gram-positive and Gram-negative bacteria and offers an attractive target for antibacterial drug development. In bacteria, fatty acid biosynthesis is mediated by FAS II system, which utilizes specific enzymes at different stages of the biosynthesis pathway. The final step in each cycle of Type II bacterial fatty acid synthesis is the 1,4-reduction of an enoyl-ACP to the corresponding acyl-ACP catalyzed by an enoyl-ACP reductase utilizing NAD(P)H as cofactor. Four different isoforms of enoyl-ACP reductase have been discovered, namely FabI, FabK, FabL and FabV. Bacteria use either one isoform, or more for fatty acid biosynthesis. Among these four subtypes, FabI has become an attractive target for antibacterial drug discovery and many compounds have already been identified as inhibitors of this enzyme (from different bacterial species). FabI, the only isoform present in Staphylococcus aureus (S. aureus or Sa) has been the target of intense drug discovery efforts for staphylococcal infections. Among other isoforms, FabV has also emerged as a potential target. B. pseudomallei have three enoyl-ACP reductases—FabI1, FabI2 and FabV. Using knockout and inhibition studies, it was shown that FabI1 (referred as FabI in this paper) is the major enoyl-ACP reductase present in B. pseudomallei.

AFN-1252, developed by Affinium Pharmaceuticals is the most advanced FabI inhibitor in clinical development. The pro-drug AFN-1720 has been developed for the IV dosing of this compound. AFN-1252 is reported to have an 10₅₀ of 14 nM for FabI from Staphylococcus aureus (SaFabI) and MIC₅₀ in the range 0.002-0.12 mg/L for S. aureus bacterial growth. AFN-1252 is potent against drug resistant staphylococci including methicillin resistant Staphylococcus aureus (MRSA) and methicillin resistant Staphylococcus epidermidis (MRSE). Triclosan is another well characterized inhibitor of FabI from multiple species including B. pseudomallei. It has been widely used as a broad spectrum biocide. Binding of Triclosan to FabI has been studied extensively using various biophysical and biochemical methods which lead to the design of more potent Triclosan analogs.

There are many reports to describe characteristics of complexes of FabI inhibitors with various FabI polypeptides from different organisms. For example, ternary complex of AFN-1252 and a cofactor bound to FabI from E. Coli (EcFabI) is reported in PDB accession code (4JQC). Similarly, ternary complexes of AFN-1252 and a cofactor bound to SaFabI (PDB: 4FS3) and CtFabI (PDB: 4Q9N) are already reported in literature. Additionally, in case of PT155, another FabI inhibitor, a ternary complex is formed with Enoyl-ACP reductase FabI from Burkholderia pseudomallei (BpmFabI) with cofactor NADH.

The reported complexes of FabI inhibitors are ternary complexes which are essentially dependent on the involvement of cofactors such as NAD(P)H. Hence, there is an unmet need for tools to understand the interactions of FabI inhibitors solely with protein residues which would contribute to the determination of active sites of FabI polypeptides, so as to utilize the model for assisting the disease treatment, e.g. the structure-based drug design.

### SUMMARY OF THE INVENTION

This invention identifies that AFN-1252 is a potent inhibitor of BpmFabI. Co-crystal structure and thermofluor data demonstrated that AFN-1252 forms a stable binary complex with BpmFabI. Kinetic studies showed that AFN-1252 can compete with NADH, but the binding is uncompetitive with crotonyl-CoA. The results of the binding studies and identification of key interactions of AFN-1252 with BpmFabI might be useful in designing and developing more potent BpmFabI inhibitors to treat melioidosis.

The present invention is directed to a crystalline structure of a binary enoyl-acyl carrier protein reductase (FabI):FabI inhibitor complex.

The present invention is directed to a related crystalline structure in which the binary complex has a space group C121 and unit cell dimensions a=134.79 Å, b=63.44 Å, c=121.84 Å and bond angles of α=γ=90°, β=107.08°.

The present invention is directed to another related crystalline structure of a binary BpmFabI: AFN-1252 complex having a protein data base accession code 4RLH.

The present invention also is directed to a method for co-crystallizing a binary enoyl-acyl carrier protein reductase (FabI) with a potential inhibitor of an FabI activity. The method comprises incubating a polypeptide having an amino acid sequence shown in SEQ ID NO: 1 with the potential FabI inhibitor to produce a binary complex with unit cell dimensions, bond angles and space group substantially identical to those of the binary crystalline complex as described herein.

The present invention is directed further to a method for identifying an inhibitor of an activity of enoyl-acyl carrier protein reductase (FabI). The method comprises generating a three-dimensional in silico model of a binary complex of FabI and a potential FabI inhibitor based at least in part on the binary complex as described herein. An interaction of the potential inhibitor with FabI within the complex to is analyzed to determine inhibitory potential.

### BRIEF DESCRIPTION OF THE DRAWINGS

So that the matter in which the above-recited features, advantages and objects of the invention, as well as others that will become clear, are attained and can be understood in detail, more particular descriptions of the invention briefly summarized above may be had by reference to certain embodiments thereof that are illustrated in the appended drawings. These drawings form a part of the specification. It is to be noted, however, that the appended drawings illustrate preferred embodiments of the invention and therefore are not to be considered limiting in their scope.

FIG. 1 is the amino acid sequence for BpmFabI (SEQ ID NO: 1).

FIG. 2 is a generic entry detailing the tabular format of large Table 1 submitted herewith as an ASCII text file. Table 1 lists the atomic structure coordinates for the amino acid sequence (SEQ ID NO: 1) of the invention derived from x-ray diffraction from a crystal of such polypeptide. The information in this generic table corresponds to the first entry in Table 1. “Record Header” refers to the row type, such as “ATOM”. “No.” designates the row number. The first “Atom Type” column describes the atom whose coordinates are measured, with the first letter in the column identifying the atom by its elemental symbol and the subsequent letter, if present, defining the location of the atom in the amino acid residue or other molecule. “Residue” and “residue number” designates the residue of the subject polypeptide. Crystallographically, “X, Y, Z” define that the atomic position of the atom measured. “Occ” is an occupancy factor that refers to the fraction of the molecules in which each atom occupies the position specified by the coordinates. A value of “1” indicates that each atom has the same conformation, i.e., the same position, in all molecules of the crystal. “B” is a thermal factor that is related to the root mean square deviation in the position of the atom around the given atomic coordinate. The ‘Chain ID’ column identifies each protein and ligand molecule present in the asymmetric unit using letters such as A, B, C, D and water molecules are identified by letter F. In the last “Atom Type” column, letter in the column identifies the atom by its elemental symbol.

FIG. 3 depicts the chemical structure of AFN-1252.

FIG. 4 is a dose-response curve for the inhibition of BpmFabI by AFN-1252.

FIG. 5A illustrates the mechanism of inhibition of BpmFabI by AFN-1252 at 300 mM crotonyl-CoA and different concentrations of NADH. The concentrations of AFN-1252 were: 0 nM (), 2.5 nM (□), 5 nM (▴), 10 nM (▾), 20 nM (A) and 40 nM (♦)

FIG. 5B illustrates the mechanism of inhibition of BpmFabI by AFN-1252 at 375 mM NADH and different concentrations of crotonyl-CoA. The concentrations of AFN-1252 were:

0 nM (), 5 nM (□), 20 nM (▴), 40 nM (▾) and 80 nM (Å)

FIG. 6 are thermal melting curves of BpmFabI alone and in presence of AFN-1252 and Triclosan

FIG. 7 is a ribbon representation of the monomer structure of BPMFabI (cyan) shown with the bound AFN-1252 (yellow) compound. The monomer composed of seven α helices and seven β strands is depicted in the figure. The figure was prepared using PyMol.

FIG. 8 is a ribbon diagram of BpmFabI tetrameric assembly viewed down one of 2-fold of the non crystallographic 222-fold axis. Each monomer along with the bound AFN-1252 is shown in different color.

FIG. 9 shows the active site structure of AFN-1252 bound to BpmFabI. The interacting residues seen within 3.6 Å from AFN-1252 are labelled. Hydrogen bonds are represented as dotted lines and water molecules as red spheres.

FIG. 10 is a cartoon diagram showing the superposed structures of BpmFabI (magenta) SA FabI (yellow) and E. coli FabI (green). The bound NAD/NADP and AFN-1252 ligands are also shown. The flexible loop (residues T194/K199) in BpmFabI was found protruding away from the active site.

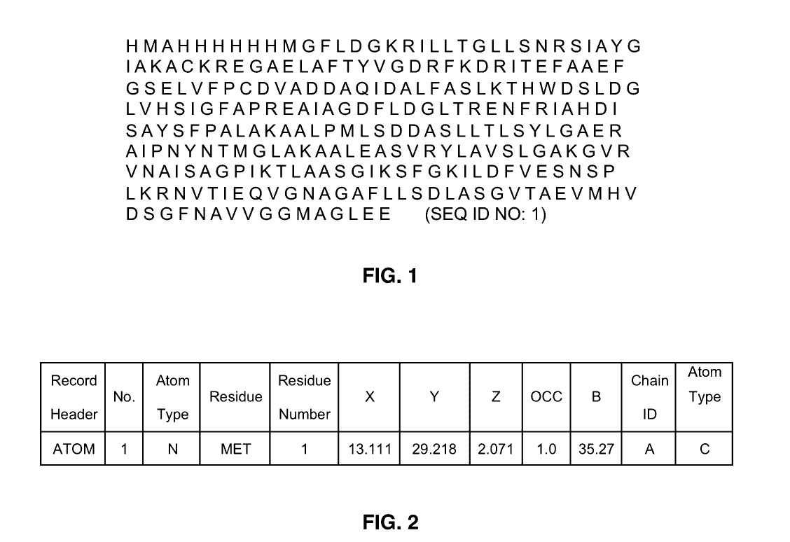

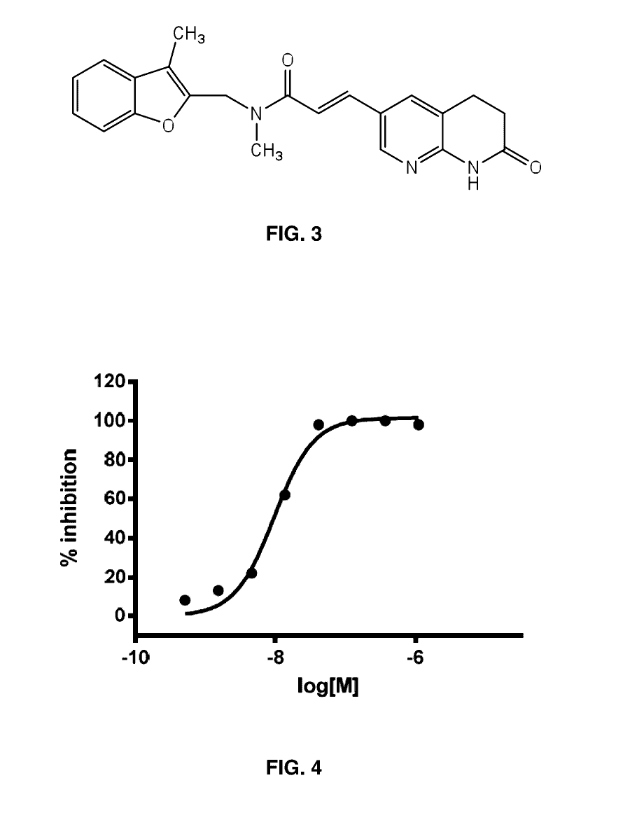

### DETAILED DESCRIPTION OF THE INVENTION

Unless defined otherwise, all technical and scientific terms used herein have the same meaning as is commonly understood by one of skill in art to which the subject matter herein belongs. As used in the specification and the appended claims, unless specified to the contrary, the following terms have the meaning indicated in order to facilitate the understanding of the present invention.

The singular forms “a”, “an” and “the” encompass plural references unless the context clearly indicates otherwise.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of the foregoing.

The term “complex” refers to a non-covalent association between at least two moieties (e.g. chemical or biochemical) that have an affinity for one another. Examples of complexes include association between antigen/antibodies, lectin/avidin, target polynucleotide/probe oligonucleotide, antibody/anti-antibody, receptor/ligand, enzyme/ligand, polypeptide/polypeptide, polypeptide/polynucleotide, polypeptide/co-factor, polypeptide/substrate, polypeptide/inhibitor, polypeptide/small molecule, and the like. “Member of a complex” refers to one moiety of the complex, such as an antigen or ligand. “Protein complex” or “polypeptide complex” refers to a complex comprising at least one polypeptide. The term “complex” is considered to be “binary complex” if the said non-covalent association is between two moieties.

The term “druggable region”, when used in reference to a polypeptide, nucleic acid, complex and the like, refers to a region of the molecule which is a target or is a likely target for binding a modulator. For a polypeptide, a druggable region generally refers to a region wherein several amino acids of a polypeptide would be capable of interacting with a modulator or other molecule. For a polypeptide or complex thereof, exemplary druggable regions including binding pockets and sites, enzymatic active sites, interfaces between domains of a polypeptide or complex, surface grooves or contours or surfaces of a polypeptide or complex which are capable of participating in interactions with another molecule. In certain instances, the interacting molecule is another polypeptide, which may be naturally-occurring. In other instances, the druggable region is on the surface of the molecule.

Druggable regions may be described and characterized in a number of ways. For example, a druggable region may be characterized by some or all of the amino acids that make up the region, or the backbone atoms thereof, or the side chain atoms thereof (optionally with or without the alpha carbon atoms). Alternatively, in certain instances, the volume of a druggable region corresponds to that of a carbon based molecule of at least about 200 amu and often up to about 800 amu. In other instances, it will be appreciated that the volume of such region may correspond to a molecule of at least about 600 amu and often up to about 1600 amu or more.

Alternatively, a druggable region may be characterized by comparison to other regions on the same or other molecules. For example, the term “affinity region” refers to a druggable region on a molecule (such as a polypeptide of the invention) that is present in several other molecules, in so much as the structures of the same affinity regions are the same so that they are expected to bind the same or related structural analogs. An example of an affinity region is an ATP-binding site of a protein kinase that is found in several protein kinases (whether or not of the same origin). The term “selectivity region” refers to a druggable region of a molecule that may not be found on other molecules, in so much as the structures of different selectivity regions are sufficiently different so that they are not expected to bind the same or related structural analogs. An exemplary selectivity region is a catalytic domain of a protein kinase that exhibits specificity for one substrate. In certain instances, a single modulator may bind to the same affinity region across a number of proteins that have a substantially similar biological function, whereas the same modulator may bind to only one selectivity region of one of those proteins.

Continuing with examples of different druggable regions, the term “undesired region” refers to a druggable region of a molecule that upon interacting with another molecule results in an undesirable affect. For example, a binding site that oxidizes the interacting molecule (such as P-450 activity) and thereby results in increased toxicity for the oxidized molecule may be deemed an “undesired region”. Other examples of potential undesired regions includes regions that upon interaction with a drug decrease the membrane permeability of the drug, increase the excretion of the drug, or increase the blood brain transport of the drug. It may be the case that, in certain circumstances, an undesired region will no longer be deemed an undesired region because the effect of the region will be favorable, e.g., a drug intended to treat a brain condition would benefit from interacting with a region that resulted in increased blood brain transport, whereas the same region could be deemed undesirable for drugs that were not intended to be delivered to the brain.

The term “polypeptide”, and the terms “protein” and “peptide” which are used interchangeably herein, refers to a polymer of amino acids. Exemplary polypeptides include gene products, naturally-occurring proteins, homologs, orthologs, paralogs, fragments, and other equivalents, variants and analogs of the foregoing.

The term “polypeptide of the invention” refers to a polypeptide comprising the amino acid sequence set forth in SEQ ID NO: 1, or an equivalent or fragment thereof, e.g., a polypeptide comprising a sequence consisting of, or consisting essentially of, the amino acid sequence set forth in SEQ ID NO: 1. Polypeptides of the invention include polypeptides comprising (i) all or a portion of the amino acid sequence set forth in SEQ ID NO: 1; (ii) the amino acid sequence set forth in SEQ ID NO: 1 with 1 to about 2, 3, 5, 7, 10, 15, 20, 30, 50, 75 or more conservative amino acid substitutions; (iii) an amino acid sequence that is at least 60%, 70%, 80%, 90%, 95%, 96%, 97%, 98%, or 99% identical to SEQ ID NO: 1; (iii) an amino acid sequence that is at least 40%, 45%, 50%, 55%, 60%, 65%, 70%, 75%, 80%, 85%, 90%, 95%, or 98% identical to SEQ ID NO: 1. Polypeptides of the invention also include homologs, e.g., orthologs and paralogs, of SEQ ID NO: 1.

The term “in silico” refers to the utilization of computer modeling or computer simulation in crystalline structure analysis and drug design. Computer systems, hardware, software, algorithms, etc. on which in silico analysis is performed are well-known and standard in the art.

In one embodiment, the present invention provides a crystalline structure of a binary enoyl-acyl carrier protein reductase (FabI):FabI inhibitor complex.

In certain embodiments, the present invention provides a binary enoyl-acyl carrier protein reductase (FabI):FabI inhibitor complex that has unit cell dimensions a=134.79 Å, b=63.44 Å, c=121.84 Å, α=γ=90°, β=107.08° with a space group C121.

In certain embodiments, the present invention provides a crystalline structure of a binary enoyl-acyl carrier protein reductase (FabI):FabI inhibitor complex where the FabI is a polypeptide comprising an amino acid sequence shown in SEQ ID NO: 1 or an amino acid sequence having about 95% identity with the amino acid sequence shown in SEQ ID NO: 1.

In certain embodiments, the present invention provides a crystalline structure of a binary enoyl-acyl carrier protein reductase (FabI):FabI inhibitor complex where the polypeptide is at least 90% pure in its non-crystalline form.

In certain embodiments, the present invention provides a crystalline structure of a binary enoyl-acyl carrier protein reductase (FabI):FabI inhibitor complex where the crystalline structure is defined by a substantial portion of atomic coordinates shown in Table 1.

In certain embodiments, the present invention provides a crystalline structure of a binary enoyl-acyl carrier protein reductase (FabI):FabI inhibitor complex where the FabI inhibitor is AFN-1252.

In certain embodiments, the present invention provides a crystalline structure of a binary enoyl-acyl carrier protein reductase (FabI):FabI inhibitor complex where the enoyl-acyl carrier protein reductase is from Burkholderia pseudomallei (BpmFabI).

In certain embodiments, the present invention provides a crystalline structure of a binary enoyl-acyl carrier protein reductase (FabI):FabI inhibitor complex that has a protein data base accession code 4RLH.

In an aspect of these embodiments, the present invention provides a crystalline structure of a binary enoyl-acyl carrier protein reductase (FabI):FabI inhibitor complex comprising the amino acid sequence set forth in SEQ ID NO: 1; where the crystalline structure comprises unit cell dimensions a=134.79 Å, b=63.44 Å, c=121.84 Å and bond angles of α=γ=90°, β=107.08° with a space group C121.

In another aspect of these embodiments, the present invention provides a crystalline structure of a binary enoyl-acyl carrier protein reductase (FabI):FabI inhibitor complex comprising the amino acid sequence having at least about 95% identity with the amino acid sequence set forth in SEQ ID NO: 1; where the crystal comprises unit cell dimensions a=134.79 Å, b=63.44 Å, c=121.84 Å and bond angles of α=γ=90°, β=107.08° with a space group C121.

In another aspect of these embodiments, the present invention provides a crystalline structure of a binary enoyl-acyl carrier protein reductase (FabI):FabI inhibitor complex where the polypeptide is at least 90% pure in its non-crystalline form; where the crystal comprises unit cell dimensions a=134.79 Å, b=63.44 Å, c=121.84 Å and bond angles of α=γ=90°, β=107.08° with a space group C121.

In certain preferred embodiments, the present invention provides a crystalline FabI from organism Burkholderia pseudomallei wherein the crystal comprises unit cell dimensions a=134.79 Å, b=63.44 Å, c=121.84 Å, α=γ=90°, β=107.08° with a space group C121.

In certain preferred embodiments, the present invention provides a crystalline FabI from organism Burkholderia pseudomallei wherein the crystal is in binary complex with a FabI inhibitor, preferably AFN-1252.

In another embodiment, the present invention provides a crystalline structure of a binary BpmFabI: AFN-1252 complex having a protein data base accession code 4RLH.

In yet another embodiment, the present invention provides a method for co-crystallizing a binary enoyl-acyl carrier protein reductase (FabI) with a potential inhibitor of an FabI activity, comprising the step of incubating a polypeptide having an amino acid sequence shown in SEQ ID NO: 1 with the potential FabI inhibitor to produce a binary complex with unit cell dimensions, bond angles and space group substantially identical to those of the binary crystalline complex as described supra.

In certain embodiments, the present invention provides a method for co-crystallizing a binary enoyl-acyl carrier protein reductase (FabI) with a potential inhibitor of an FabI activity where co-crystallizing occurs in the absence of a cofactor.

In certain embodiments, the present invention provides a method for co-crystallizing a binary enoyl-acyl carrier protein reductase (FabI) with a potential inhibitor of an FabI activity where co-crystallizing occurs in the absence of the cofactor NADH or NADPH.

In certain embodiments, the present invention provides a method for co-crystallizing a binary enoyl-acyl carrier protein reductase (FabI) with a potential inhibitor of an FabI activity where the polypeptide has an amino acid sequence having about 95% identity with the amino acid sequence shown in SEQ ID NO: 1.

In aspects of these embodiments, the present invention provides a method for co-crystallizing a binary enoyl-acyl carrier protein reductase (FabI) with a potential inhibitor of an FabI activity where the crystalline FabI has unit cell dimensions a=134.79 Å, b=63.44 Å, c=121.84 Å, α=γ=90°, β=107.08° with a space group C121 in the absence of cofactors.

In aspects of these embodiments, the present invention provides a method for co-crystallizing a binary enoyl-acyl carrier protein reductase (FabI) with a potential inhibitor of an FabI activity where the crystalline FabI has unit cell dimensions a=134.79 Å, b=63.44 Å, c=121.84 Å, α=γ=90°, β=107.08° with a space group C121 in the absence of NADH.

In aspects of these embodiments, the present invention provides a method for co-crystallizing a binary enoyl-acyl carrier protein reductase (FabI) with a potential inhibitor of an FabI activity where the crystalline FabI has unit cell dimensions a=134.79 Å, b=63.44 Å, c=121.84 Å, α=γ=90°, β=107.08° with a space group C121 in the absence of NADPH.

In aspects of these embodiments, the present invention provides a method for co-crystallizing a binary enoyl-acyl carrier protein reductase (FabI) with AFN-1252 where the crystalline FabI having unit cell dimensions a=134.79 Å, b=63.44 Å, c=121.84 Å and bond angles of α=γ=90°, β=107.08° with a space group C121 comprising placing the crystalline FabI in a solution comprising the AFN-1252.

In yet another embodiment, the present invention provides a FabI:FabI inhibitor binary crystalline complex produced by the method described supra.

In yet another embodiment, the present invention provides a method for identifying an inhibitor of an activity of enoyl-acyl carrier protein reductase (FabI), comprising the steps of generating a three-dimensional in silico model of a binary complex of FabI and a potential FabI inhibitor based at least in part on the binary complex as described supra; and analyzing an interaction of the potential inhibitor with FabI within the complex to determine inhibitory potential.

In certain embodiments, the present invention provides a method for identifying an inhibitor of an activity of enoyl-acyl carrier protein reductase (FabI), comprising the steps of inputting into the model a set of atomic structure coordinates for atoms of amino acid residues from druggable regions of FabI; inputting a set of atomic structure coordinates for the potential inhibitor; performing a fitting operation between the potential inhibitor and the druggable region of FabI; and quantifying the association between the potential inhibitor and the druggable region of FabI, thereby determining the inhibitory potential.

In certain embodiments, the present invention provides a method for identifying an inhibitor of an activity of enoyl-acyl carrier protein reductase (FabI) as described supra further comprising screening the potential inhibitor for inhibition of the activity of FabI.

In certain embodiments, the present invention provides a method for identifying an inhibitor of an activity of enoyl-acyl carrier protein reductase (FabI) where the atomic structures coordinates for the atoms comprising the druggable regions of FabI are shown in Table 1.

In certain embodiments, the present invention provides a method for identifying an inhibitor of an activity of enoyl-acyl carrier protein reductase (FabI) where the FabI is from Burkholderia pseudomallei (Bpm) and the identified inhibitor is a drug for a BpmFabI associated disease.

In certain embodiments, the present invention provides a method for identifying an inhibitor of an activity of enoyl-acyl carrier protein reductase (FabI) where the FabI is from Burkholderia pseudomallei (Bpm) and the identified inhibitor is a drug for melioidosis.

In an aspect of these embodiments, the present invention provides a method for identifying an inhibitor of an activity of enoyl-acyl carrier protein reductase (FabI) where the FabI crystal comprises unit cell dimensions a=134.79 Å, b=63.44 Å, c=121.84 Å, α=γ=90°, β=107.08° with a space group C121 to treat a Burkholderia associated disease.

In an aspect of these embodiments, the present invention provides a method for identifying an inhibitor of an activity of enoyl-acyl carrier protein reductase (FabI) where the FabI crystal comprises unit cell dimensions a=134.79 Å, b=63.44 Å, c=121.84 Å, α=γ=90°, β=107.08° with a space group C121 to treat melioidosis.

In certain embodiments, the present invention provides a method for identifying or designing an inhibitor of the activity of BpmFabI for the treatment of BpmFabI associated diseases, particularly melioidosis, including: a) supplying a computer modeling application with a set of structure coordinates of a complex, including at least a portion of a druggable region from a BpmFabI; b) supplying the computer modeling application with a set of structure coordinates of a chemical entity; and c) determining whether the chemical entity is expected to bind to the complex, wherein binding to the complex is indicative of potential modulation of the activity of a BpmFabI.

In certain embodiments, the present invention provides a method for identifying or designing an inhibitor of the activity of BpmFabI, supplying a computer modeling application with a set of structure coordinates of a complex wherein the complex comprises at least a portion of a druggable region of a BpmFabI; supplying the computer modeling application with a set of structure coordinates for a chemical entity; evaluating the potential binding interactions between the chemical entity and active site of the molecular complex; structurally modifying the chemical entity to yield a set of structure coordinates for a modified chemical entity, and determining whether the modified chemical entity is expected to bind to the complex, wherein binding to the complex is indicative of potential modulation of the BpmFabI.

In certain embodiments, the present invention provides a method for obtaining structural information about a molecule or a molecular complex of unknown structure comprising: (a) crystallizing the molecule or molecular complex; (b) generating an x-ray diffraction pattern from the crystallized molecule or molecular complex; (c) applying at least a portion of the structure coordinates set forth in Table 1 to the x-ray diffraction pattern to generate a three-dimensional electron density map of at least a portion of the molecule or molecular complex whose structure is unknown.

Provided herein are crystalline structures comprising an enzyme enoyl-acyl carrier protein reductase (FabI) from, for example, Burkholderia pseudomallei (BpmFabI) as solved and described herein. The BpmFabI crystal provides information about the structure of the polypeptide comprising the FabI, such as shown in SEQ ID NO: 1, and druggable regions or domains and the like contained therein, all of which could help in the rational design and development of new FabI inhibitors to treat diseases associated with B. pseudomallei. Also provided are methods utilizing the crystalline structures to design modulators of one or more of their biological activities. In particular, the present invention provides a use of crystalline structure of BpmFabI to design therapeutic and diagnostic molecules. Moreover, modulators, inhibitors, agonists or antagonists against the polypeptides comprising the crystalline structure, or biological complexes containing them, or orthologous thereto, may be used to treat any disease or other treatable condition of a patient, including humans and animals, and particularly to treat a disease caused by or associated with Burkholderia pseudomallei.

The present invention demonstrates that AFN-1252 is a potent inhibitor of BpmFabI. Co-crystal structure and thermofluor data demonstrated that AFN-1252 forms a stable binary complex with BpmFabI. Kinetic studies show that AFN-1252 can compete with NADH, but the binding is uncompetitive with crotonyl-CoA. The results of the binding studies and identification of key interactions of AFN-1252 with BpmFabI are useful in designing and developing more potent BpmFabI inhibitors to treat, for example, but not limited to, melioidosis.

The following examples are used to illustrate the certain aspects and embodiments of the present invention in more details, and are not intended to limit the invention in any way.

### Example 1

### Inhibitory Potential of AFN-1252 on BpmFabI

### a) Expression and Purification of BpmFabI Isoform 1

Burkholderia pseudomallei FabI (Isoform1) gene corresponding to amino acids 1 to 263 was synthesized and subcloned into pET21a vector. Transformants of E. coli BL21DE3 star strain containing pET21a-BpmFabI (isoform 1) was induced with 0.5 mM IPTG for 16 h at 18° C. Cell pellet was washed with lysis buffer (20 mM Tris pH 8.0, 500 mM NaCl, 1 mM PMSF and 5 mM Imidazole) and treated with 50 μg/ml lysozyme for 30 min. Cells were lysed by sonication and the cell debris clarified by centrifugation at 12000 rpm for 40 min at 4° C. The supernatant was bound to Ni-NTA beads pre-equilibrated with 20 mM Tris pH 8.0, 500 mM NaCl and 5 mM imidazole buffer, and eluted with 500 mM imidazole. The fractions containing BpmFabI passed through Superdex-75 column in 20 mM Tris pH 8.0, 500 mM NaCl, 5 mM Imidazole.

### b) BpmFabI Enzyme Inhibition Assay for AFN-1252

The potency of AFN-1252 to inhibit BpmFabI was evaluated in a spectrophotometric assay by monitoring the oxidation of the cofactor NADH. Buffer used for the assay was 30 mM PIPES, pH 6.8, containing 150 mM NaCl and 1 mM EDTA. The Michaelis Menton constant (Km) of crotonyl-CoA was determined from the enzyme activity at increasing concentrations of this cofactor (data not shown). AFN-1252 was pre-incubated with BpmFabI for 30 minutes and the reaction was started by adding substrate mix containing crotonyl-CoA (300 μM) and NADH (375 μM). The oxidation of NADH was monitored by following the decrease of absorbance at 340 nm. IC₅₀ value was determined by fitting the dose-response data to sigmoidal dose response (variable slope) curve using Graphpad Prism software V4. To determine the mechanism of binding, kinetic studies were carried out at different concentrations of inhibitor and varying the concentration of NADH at a fixed concentration of crotonoyl CoA (300 μM) and also by varying the concentrations of crotonoyl CoA keeping NADH concentration fixed at 375 μM. Lineweaver-Burk plots were subsequently generated to determine the mechanism of binding of AFN-1252 to BpmFabI. Since AFN-1252 is reported to be a specific inhibitor of Gram-positive S. aureus, it was surprising to see that it is active against Gram-negative B. pseudomallei bacterium.

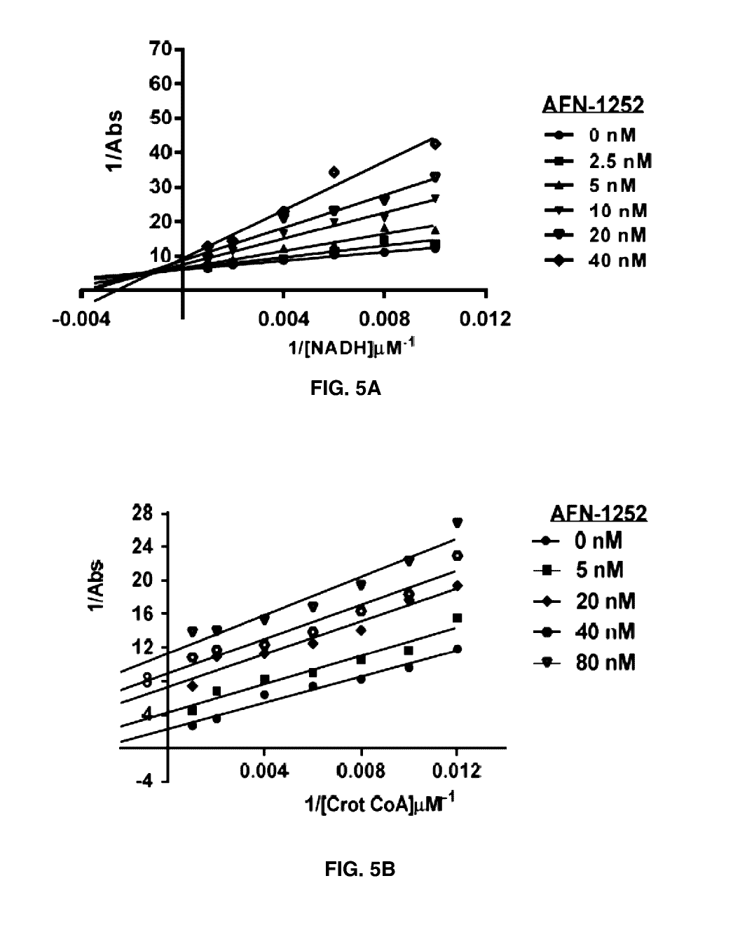

As shown in FIG. 4, AFN-1252 inhibited BpmFabI with an IC₅₀ of 9.6 nM. Lineweaver-Burk plots were generated at different concentrations of AFN-1252 to understand the mechanism of inhibition of AFN-1252 in the presence of NADH and crotonyl-CoA. The concentration of either crotonyl-CoA or NADH was varied, keeping the other constant. The Lineweaver-Burk plot for AFN-1252 binding to BpmFabI at 300 μM crotonyl-CoA and varying concentrations of NADH is shown in FIG. 5A. Increase in Km (seen as decrease in 1/[NADH]) with increasing concentrations of AFN-1252, and a corresponding decrease in Vmax (seen as increase in 1/Abs value) suggested mixed inhibition of BpmFabI in which AFN-1252 could bind to enzyme directly, and also to BpmFabI-NADH complex. The Lineweaver-Burk plot for AFN-1252 binding to BpmFabI at 375 uM NADH and varying concentrations of crotonyl-CoA is shown in FIG. 5B. Decrease in Km with corresponding decrease in Vmax suggested that AFN-1252 was uncompetitive with crotonyl-CoA for BpmFabI binding and the inhibitor was bound to BpmFabI-crotonyl-CoA complex.

### c) Thermofluor Assay

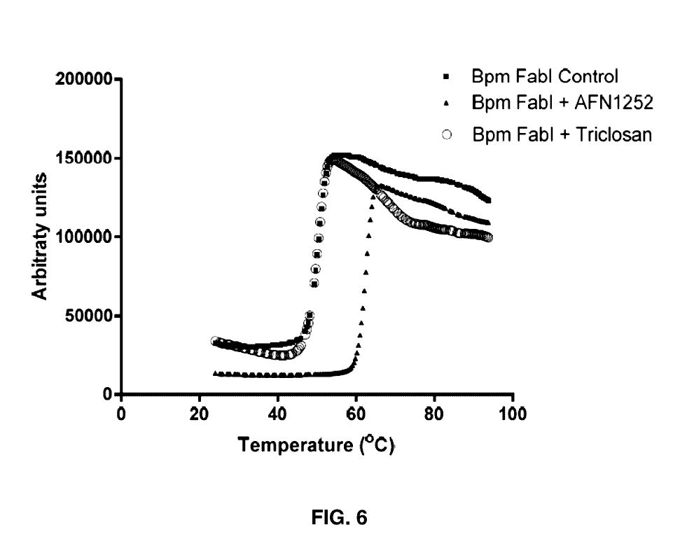

Binding of AFN-1252 to BpmFabI was also monitored by thermofluor assay. AFN-1252 stabilized the enzyme as determined by the increase in melting temperature of ˜12° C. (FIG. 6 & Table 2). NADH did not have any effect on the Tm of BpmFabI or BpmFabI:AFN-1252 complex. For comparison, a similar experiment was carried out with Triclosan. Interestingly, Triclosan stabilized BpmFabI only in the presence of NADH (ΔTm of ˜9° C.); neither NADH nor Triclosan alone stabilized the protein. This data suggests the formation of a ternary complex of BpmFabI-Triclosan-NADH, indicating that AFN-1252 binding mechanism is different as compared to that of Triclosan. The results are given in Table 2.

TABLE 2
Stabilization effect of Triclosan/AFN-1252 on BpmFabI

| Ligand | ΔTm (° C.) |
|---|---|
| NADH | 0 |
| AFN-1252 | 12.76 ± 1.26 |
| AFN-1252 + NADH | 11.61 ± 0.10 |
| Triclosan | 0 |
| Triclosan + NADH | 8.49 ± 0.32 |

### Example 2

### X-Ray Crystallography

### a) Crystallization of BpmFabI Isoform 1

Co-crystals of BpmFabI with AFN-1252 were obtained using hanging drop vapor diffusion technique. Concentrated protein (15 mg/ml) was incubated overnight with 0.5 mM AFN-1252 and 1 mM NADH in a reservoir buffer containing 0.1 M MES pH 5.0, 0.1 M NaCl, 10% PEG 3350.

### b) X-Ray Data Collection and Structure Determination

Co-crystals of BpmFabI with AFN-1252 were flash frozen at 100K using 20% glycerol as cryo-protectant. The diffraction data was collected using in-house Rigaku RU300 X-ray generator with R-AXIS IV++detector to a maximum resolution of 2.3 Å. Data indexing, integration and scaling were performed using DENZO and SCALEPACK. The structure was solved by molecular replacement (MR) method using the search model with PDB Code 3EK2. Alternate cycles of restrained refinement and manual rebuilding were performed with the programs REFMAC 5.2.0001 and Coot respectively. Five percent of the reflections were randomly excluded from the refinement to monitor the free residual-factor (R_{free}). A summary of the data reduction and structure refinement statistics is provided in Table 3.

TABLE 3
Diffraction data and structure refinement statistics of BpmFabI:
AFN-1252 Complex

| Space Group | C121 | |
|---|---|---|
| Cell Parameters (Å) | a = 134.79, b = 63.44, c = 121.84, | |
| | α = γ = 90°, β = 107.08° | |
| Resolution range (Å) | 40-2.26 | |
| Total no. reflections | 114,107 | |
| Unique reflections | 42,792 | |
| Completeness (%) | 93.11 | (89.9)a* |
| Rsym | 0.062 | (0.157) |
| I/σI | 11.8 | (2.6) |
| Multiplicity | 2.7 | (2.7) |

Refinement

| Resolution (Å) | 40-2.26 | |
|---|---|---|
| No. of reflections | 40,619 | |
| Completeness (%) | 92.48 | (79.28) |

| | |
|---|---|
| Rwork/Rfree | 0.161/0.250 |

r.m.s. deviations

| | |
|---|---|
| Bond lengths (Å) | 0.015 |
| Bond angles (°) | 1.855 |

Ramachandran plot

| | |
|---|---|
| Residues in the most favored region | 96.5 |
| (%) | |
| Residues in the allowed region (%) | 3.5 |

a*Values corresponding to the outermost shell are given within parentheses.

### Example 3

### Analysis of X-Ray Structure of BpmFabI

### a) Monomer and Quaternary Structures of the BpmFabI

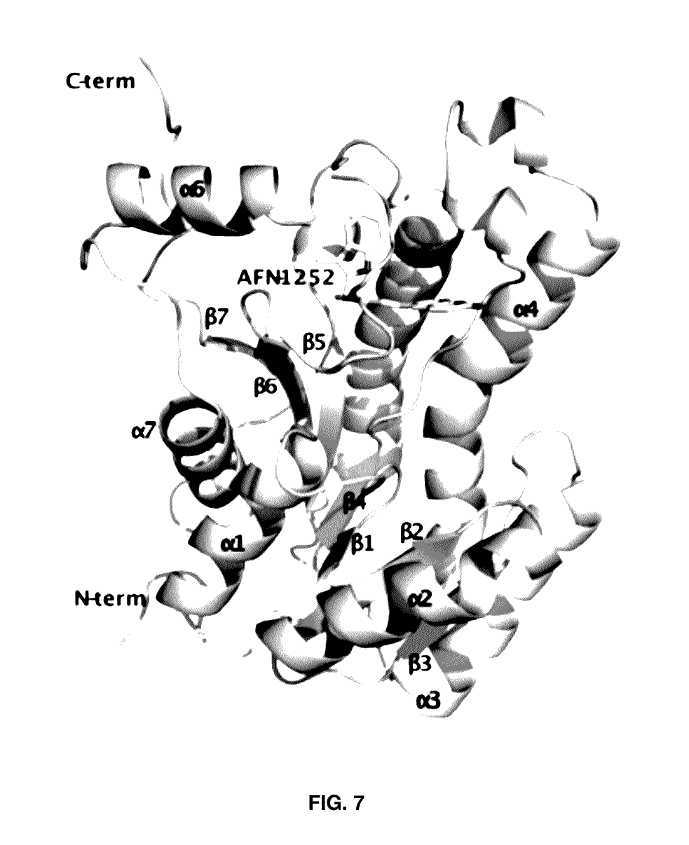

The BpmFabI monomer contains a single domain composed of a seven-stranded parallel β-sheet (β1, β2, β3, β4, β5 β6, β7) sandwiched by three α-helices from the top (α1, α2, α3) and three from the bottom (α4, α5, α6). Another helix, α7 is located at the C terminal tail of the protein (FIG. 7). The overall fold is common to dinucleotide-binding FabI enzymes and resembles the typical Rossmann fold. The structural superposition showed an r.m.s. deviation of 1.047 Å, 0.87 Å and 1.07 Å respectively for Bpm (256 Ca atoms; PDB; 3EK2), Ec (251 Ca atoms; PDB; 4JQC) and ScFabI (242 Ca atoms; PDB; 4FS3) crystal structures.

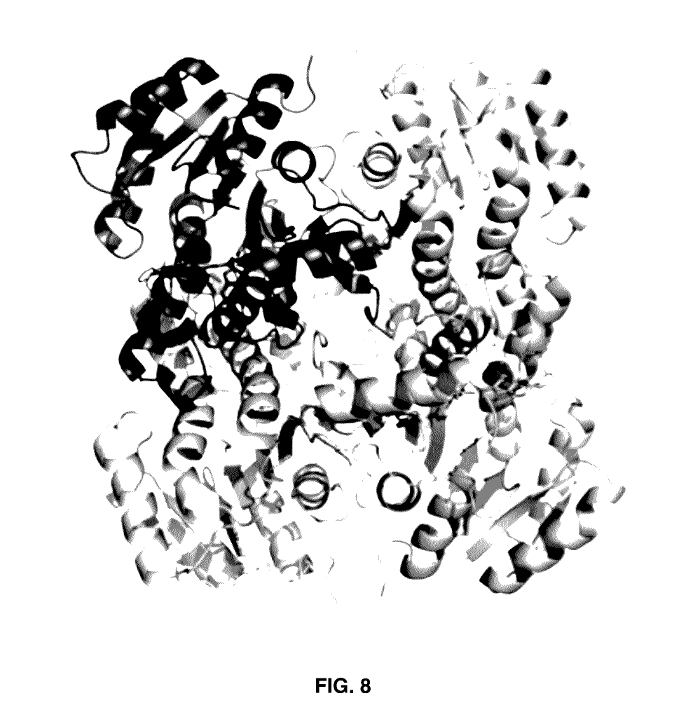

The crystallographic asymmetric unit consists of four protomers arranged as a tetramer with an approximate 222 symmetry (FIG. 8). The elements involved in the tetramer formation include the β7 strand, helices α4 and α5, and the C-terminal tail which cross over to the neighboring monomer. The helices α4 and α5 from the two monomers interact with each other to create a strong dimer. The β7 strand from the seven-stranded n-sheet of each dimer associates to form an extended 14 stranded β-sheet, which facilitate the formation of a stable tetrameric assembly. This larger assembly may represent the biologically active form of BpmFabI. This is further supported by analytical gel filtration experiments which indicated a tetrameric protein in solution.

### b) Binding Site Structure of AFN-1252 in BpmFabI

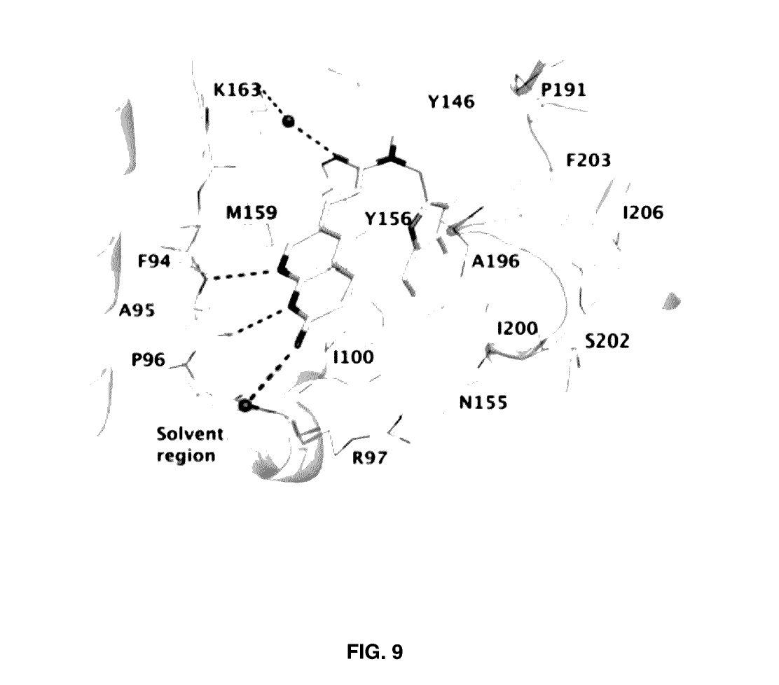

The electron density for AFN-1252 was clearly visible in the active site cavity in all four subunits. The flexible loop containing protein residues 194 to 204 accommodates the AFN-1252 molecule in the binding pocket. AFN-1252 binding to BpmFabI is mediated through multiple chemical interactions (FIG. 9). The tetrahydronaphthyridone moiety of the molecule makes two hydrogen bonds to the main chain atoms of Ala95. A water mediated interaction is observed between the main chain NH atom of Arg97 and the —CO group of napthyridinone ring. The amide —CO group of the inhibitor also forms hydrogen bonds with the side chain of Tyr156 and a water mediated interaction with Lys163 side chain. The 3-methylbenzofuran moiety is located in a hydrophobic pocket created by the side chains of Ile100, Tyr146, Tyr156, Pro191, Phe203 and Ile206.

Comparison of crystal structures of AFN-1252:BpmFabI complex with the apo-BpmFabI (PDB-3EK2) revealed no major conformational changes except in the active site region. Near the active site, the loop containing residues Thr194-Lys199 adopts ordered conformation as a result of AFN-1252 binding, whereas the same loop is highly disordered in the apo structure. Thus the change in loop conformation appears to have been induced by the AFN-1252 binding. Further, involvement of this flexible loop in the formation of binary/ternary complexes across FabIs is well established.

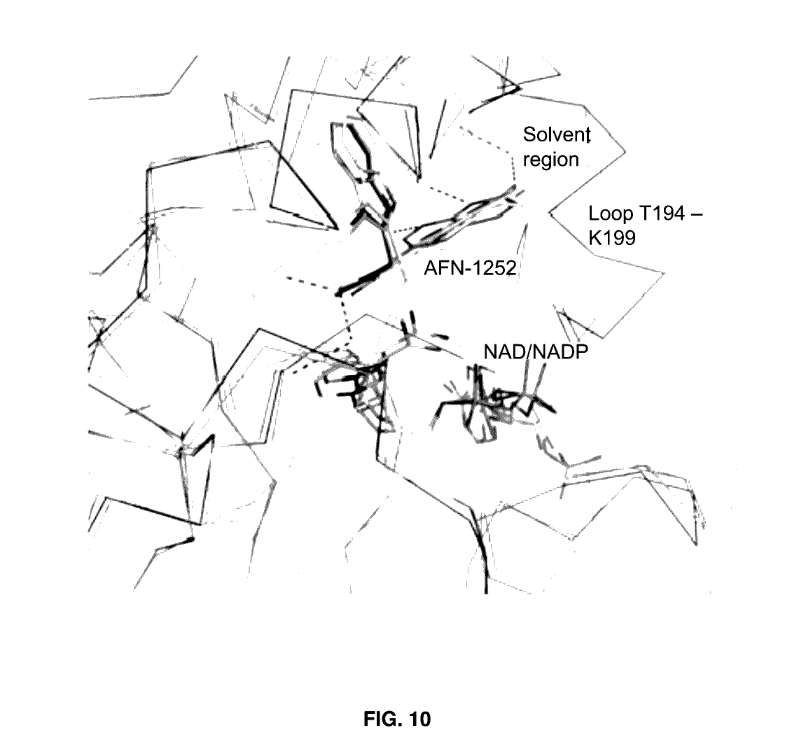

From this analysis, it was found that AFN-1252 formed binary complex with BpmFabI which was compared with reported ternary complexes of AFN-1252 and co-factor bound to Ec (PDB-4JQC) and SaFabI (PDB-4FS3) wherein strong hydrogen bonds and π-π contacts were observed between the co-factor and AFN-1252. Comparison of AFN-1252 bound binary and ternary complex structures revealed no major conformational changes due to the presence or absence of the co-factor in the active site (FIG. 10). Thus the BpmFabI binary complex structure establishes that an inhibitor binding is feasible in the absence of the cofactor. This finding suggests that inhibitors for BpmFabI can be designed by considering interactions solely with protein residues. The binary BpmFabI:AFN-1252 comples was deposited and assigned protein data base accession code 4RLH.

The co-crystal structure of AFN-1252: BpmFabI complex shows that the inhibitor binds to the enzyme as a binary complex. This binding mode is different from the reported ternary complex of EcFabI/SaFabI with Triclosan and NADH. Binding of AFN-1252 to FabI is mediated by hydrogen bonding interactions and further stabilized by π-π interactions. AFN-1252 stabilized the enzyme in the absence of NADH which was in contrast with Triclosan wherein stabilization effect on BpmFabI was observed only when both NADH and Triclosan were present. Mixed mode of inhibition observed in kinetic studies also shows that AFN-1252 can compete with NADH for BpmFabI binding site.

The following references provide background to the invention described herein.
1. Bergler, et al, J. Biol. Chem. (1994), 269, 5493-5496
2. Heath et al, J. Biol. Chem. (1996), 271, 1833-1836
3. Hayden H S, PLoS One (2012) 7(5): e36507, 1-14
4. Limmathurotsakul D et al, Emerg Infect Dis (2006-2010), 18(2): 325-327
5. Wiersinga W J et al, N Engl J Med (2012), 367(11): 1035-44
6. Limmathurotsakul D et al, Br Med Bull (2011), 99: 125-139
7. Cheng A C et al, Am J Trop Med Hyg (2013), 88(3): 411-413
8. Dance D, Int J Antimicrob Agents (2014), 43(4): 310-318
9. Estes D M et al, Expert Rev Anti Infect Ther (2010), 8(3): 325-38)
10. Schweizer H P, Future Microbiol (2012), 7(12): 1389-99
11. Sarovich D S et al, Infect Drug Resist (2012), 5: 129-32
12. Moore R A et al, Antimicrob Agents Chemother (1999), 43(3): 465-70
13. Campbell J W et al, Annu Rev Microbiol (2001), 55: 305-332
14. Parsons J B et al, Curr Opin Microbiol (2011), 14: 544-49
15. Wang Y et al, Chem Med Chem (2013), 8(10): 1589-1608
16. Cummings J E et al, Antimicrob Agents and Chemother (2014), 58(2): 931-935
17. Lu H et al, Acc Chem Res (2008), 41(1): 11-20
18. Heath R J et al, J Biol Chem (1998), 273: 30316-30320
19. Takhi M et al, Eur J Med Chem (2014), 84: 382-394
20. Miller W H et al, J Med Chem (2002) 45: 3246-56
21. Tsuji B T et al, Biochemistry (2010), 49: 1281-89
22. Tsuji B T et al., J Chemother (2013), 25(1):32-35.
23. Kumar K, et al, J Antimicrob Chemother (2013), 68 (7): 1465-1470
24. Kaplan N et al, Eur J Pharm Sci (2013), 50(3-4): 440-6
25. Kaplan N et al, Antimicrob Agents Chemother (2012), 56: 5865-5874
26. Karlowsky J A et al, Antimicrob Agents Chemother (2009), 53: 3544-48
27. Yao J et al, J Biol Chem (2013), 288: 36261-36271.
28. Escalada et al., J Antimicrob Chemother (2005), 55:879-882.
29. Heath et al., J Biol Chem (1999), 274:11110-14.
30. Gerusz V et al, J Med Chem (2012), 55: 9914-28
31. Krishnamurthy Narasimha Rao et al., Protein Sci. (2015), 24(5): 832-40
32. Schiebel, J et al, (2014) J. Biol. Chem. 289: 15987.
33. Henry Pauls et al., International patent publication WO2008098374 A1.
34. Liu N et al., J Antimicrob Chemother (2011), 66: 564-573.
35. Wiegand et al., Nature Protoc (2008), 3(2):163-175.
36. Lee et al., Diagn Microbiol Infect Dis (2007), 53(3):263-70.
37. Otwinowski Z et al., Academic Press Inc. NY (1997), 276: 307-326.
38. Murshudov G N et al., Acta Crystallogr D Biol Crystallogr (1997), 53: 240-55.
39. Beveridge T J J Bacteriol (1999), 181(16): 4725-4733.

All publications and patent applications mentioned herein are hereby incorporated by reference in their entirety as if each individual publication or patent was specifically and individually indicated to be incorporated by reference. In case of conflict, the present application, including any definitions herein, will control. While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

What is claimed is:

1. A crystalline structure of a binary enoyl-acyl carrier protein reductase (FabI):FabI inhibitor complex.

2. The crystalline structure of claim 1, wherein said binary complex has a space group C121 and unit cell dimensions a=134.79 Å, b=63.44 Å, c=121.84 Å and bond angles of α=γ=90°, β=107.08°.

3. The crystalline structure of claim 1, wherein said FabI is a polypeptide comprising:
(a) an amino acid sequence shown in SEQ ID NO: 1; or
(b) an amino acid sequence having about 95% identity with the amino acid sequence shown in SEQ ID NO: 1.

4. The crystalline structure of claim 3, wherein the polypeptide is at least 90% pure in its non-crystalline form.

5. The crystalline structure of claim 1, wherein said crystalline structure is defined by a substantial portion of atomic coordinates shown in Table 1.

6. The crystalline FabI according to claim 1, wherein the FabI inhibitor is AFN-1252.

7. The crystalline structure of claim 1, wherein said enoyl-acyl carrier protein reductase is from Burkholderia pseudomallei (BpmFabI).

8. The crystalline structure of claim 1 having a protein data base accession code 4RLH.

9. A method for co-crystallizing a binary enoyl-acyl carrier protein reductase (FabI) with a potential inhibitor of an FabI activity, comprising the step of:
incubating a polypeptide having an amino acid sequence shown in SEQ ID NO: 1 with the potential FabI inhibitor to produce a binary complex with unit cell dimensions, bond angles and space group substantially identical to those of the binary crystalline complex of claim 2.

10. The method of claim 9, wherein co-crystallizing occurs in the absence of a cofactor.

11. The method of claim 10, wherein the cofactor is NADH or NADPH.

12. The method of claim 9, wherein the polypeptide has an amino acid sequence having about 95% identity with the amino acid sequence shown in SEQ ID NO: 1.

13. The FabI:FabI inhibitor binary crystalline complex produced by the method of claim 9.

14. A method for identifying an inhibitor of an activity of enoyl-acyl carrier protein reductase (FabI), comprising the steps of:
generating a three-dimensional in silico model of a binary complex of FabI and a potential FabI inhibitor based at least in part on the binary complex of claim 1; and
analyzing an interaction of the potential inhibitor with FabI within the complex to determine inhibitory potential.

15. The method of claim 14, comprising the steps of:
inputting into the model a set of atomic structure coordinates for atoms of amino acid residues from druggable regions of FabI;
inputting a set of atomic structure coordinates for the potential inhibitor;
performing a fitting operation between the potential inhibitor and the druggable region of FabI; and
quantifying the association between the potential inhibitor and the druggable region of FabI, thereby determining the inhibitory potential.

16. The method of claim 14, further comprising screening said potential inhibitor for inhibition of the activity of FabI.

17. The method of claim 15, wherein the atomic structures coordinates for the atoms comprising the druggable regions of FabI are shown in Table 1.

18. The method of claim 14, wherein the FabI is from Burkholderia pseudomallei (Bpm) and the identified inhibitor is a drug for a BpmFabI associated disease.

19. The method of claim 18, wherein the BpmFabI associated disease is melioidosis.

20. A crystalline structure of a binary BpmFabI:AFN-1252 complex having a protein data base accession code 4RLH.


SEQUENCE LISTING (1 sequence)

SEQ ID NO: 1 - protein, 272 aa, Burkholderia pseudomallei
  enoyl-acyl carrier protein reductase (FabI) sequence
HMAHHHHHHMGFLDGKRILLTGLLSNRSIAYGIAKACKREGAELAFTYVGDRFKDRITEFAAEFGSELVFPCDVADDAQIDALFASLKTHWDSLDGLVHSIGFAPREAIAGDFLDGLTRENFRIAHDISAYSFPALAKAALPMLSDDASLLTLSYLGAERAIPNYNTMGLAKAALEASVRYLAVSLGAKGVRVNAISAGPIKTLAASGIKSFGKILDFVESNSPLKRNVTIEQVGNAGAFLLSDLASGVTAEVMHVDSGFNAVVGGMAGLEE